(19) United States
(12) Patent Application Publication
(10) Pub. No.: US 2019/0320727 A1
(43) Pub. Date: Oct. 24, 2019

(54) DETACHABLE PANEL UNDERWEAR SYSTEM WITH ADJUSTABLE WAIST

(71) Applicant: Candace Bain, Anaheim, CA (US)

(72) Inventor: Candace Bain, Anaheim, CA (US)

(21) Appl. No.: 15/958,495

(22) Filed: Apr. 20, 2018

(51) Int. Cl. A41B 9/00; A41B 9/04

(52) U.S. Cl. CPC A41B 9/004; A61F 13/5605; A41B 9/04

(57) ABSTRACT

A detachable panel underwear system is disclosed. The detachable panel underwear system includes a fabric undergarment including three outer edges, the three outer edges including a first waist outer edge, a first leg outer edge and a second leg outer edge and a central gusset. The system further includes a detachable gusset pad in which the central gusset is located between the first leg outer edge and the second leg outer edge and the detachable gusset pad is disposed to attach over the central gusset.

### TECHNICAL FIELD

The present embodiments relate generally to undergarments and more specifically to underwear for women.

### BACKGROUND

Women experience menstrual cycles that are often less than pleasant. Most women bodies change and so does their menstrual cycles. When a women accidentally starts her cycle may stain the inside gusset (or female reproductive issues down there ex. Discharges). Sanitary napkins are disposable absorbent pads affixed to a user's undergarments via adhesive means integrated in the underside of the pad. These sanitary napkins are bulky and uncomfortable besides often being conspicuous through one's clothing. Wearing a form fitting outfit during one's period is rarely an option when the user chooses to wear a pad. Sanitary napkins shift as a woman walks and frequently flip upside down, reversing the adhesive side upward onto the wearer, requiring constant repositioning, (not to mention drawing even further attention to an already tenuous situation). Despite the painful prospect of a pad's adhesive coating sticking to its wearer, there is also the sweaty, itchy chafing sensation that results from wearing a pad for any extended period of time (particularly in warmer weather). Developing rashes and other irritations are not uncommon for many women who choose to wear sanitary napkins. Physical activity of any kind while wearing a sanitary napkin is often an arduous and exasperating task.

The other option besides a sanitary pad is a tampon; an absorbent plug inserted directly into a woman's body. For many, inserting a tampon is uncomfortable and in some cases, painful. The risks associated with tampons, such as Toxic Shock Syndrome (T.S.S.) and other bacterial infections, have left an uneasy feeling in many female consumers, who shun the merits of tampons despite their convenience. Those who do choose to wear tampons change them rather frequently, as fear of T.S.S. and other infections is an ever present concern for most modern women. Feeling ambivalent about either choice is the norm, and yet alternative options are hard to find. Some alternatives currently available on the market are reusable wash pads, sea sponges, and “menstrual cups”, all of which are inconvenient and unrealistic for various reasons.

Ruining underwear is only part of the humiliation suffered by many females during their monthly cycles. Oftentimes blood stains make their way through the pad and underwear and onto a woman's clothes. In terms of embarrassment, no woman can deny the sheer honor of discovering a blood stain on one's clothes and the frantic scramble to conceal it before others find out. Putting the embarrassment and humiliation aside, the cost factor of having to replace clothing as a result of staining adds insult to injury. A blood stain that has set into a fabric for a good period of time has almost no chance of coming out. And so, the unfortunate truth seems to be that discomfort, embarrassment and self-consciousness are all overwhelming themes throughout many females' menstrual cycles.

Thus, a need exists for a reliable women's underwear system to provide discreet absorption for a sustained duration of menstruation and/or incontinence and thereby increase the comfort, security, and confidence of a user and to avoid the above-mentioned problems.

### SUMMARY

A detachable panel underwear system is disclosed in one embodiment. The detachable panel underwear system includes a fabric undergarment including three outer edges, the three outer edges including a first waist outer edge, a first leg outer edge and a second leg outer edge and a central gusset. The system further includes a detachable gusset pad in which the central gusset is located between the first leg outer edge and the second leg outer edge and the detachable gusset pad is disposed to attach over the central gusset.

In another embodiment an underwear system is disclosed. The underwear system includes a fabric undergarment with three outer edges, the three outer edges including a waist band outer edge, a first leg outer edge and a second leg outer edge and a central gusset. The underwear system further includes a detachable gusset pad and a clasp adjacent to the waist band outer edge, in which the central gusset is located between the first leg outer edge and the second leg outer edge and the detachable gusset pad is disposed to attach over the central gusset.

### DRAWINGS

The following figures set forth embodiments of the invention in which like reference numerals denote like parts. Embodiments of the invention are illustrated by way of example and not by way of limitation in the accompanying figures.

FIG. 1 is perspective view of an example of underwear of the prior art;

FIG. 2 is a top plan view of an example of underwear in an embodiment of the prior art including a gusset;

FIG. 3 is a top plan view of an example of the detachable panel underwear system in an embodiment of the present invention;

FIG. 4 is a plan view of a gusset pad in an embodiment of the present invention;

FIG. 5 is a front view of the gusset pad in an embodiment of the present invention;

FIG. 6 is a plan view of an exemplary gusset pad used in the detachable panel underwear system in an embodiment of the present invention;

FIG. 7 is a plan view of an alternate gusset in an embodiment of the present invention;

FIG. 8 is a plan view of an exemplary gusset pad in another embodiment of the present invention;

FIG. 9 is a plan view of an exemplary gusset pad used in the detachable panel underwear system in an embodiment of the present invention;

FIG. 10 is a perspective view of an example of the detachable panel underwear system including an adjustable clasp in an embodiment of the present invention;

FIG. 11 is an isolation view of an example an adjustable clasp of the detachable panel underwear system in an embodiment of the present invention;

FIG. 12 is a plan view of an example of the detachable panel underwear system including an adjustable clasp in an embodiment of the present invention.

### DETAILED DESCRIPTION

The description set forth below in connection with the appended drawings is intended as a description of various configurations and is not intended to represent the only configurations in which the concepts and features described herein may be practiced. The following description includes specific details for the purpose of providing a thorough understanding of various concepts. However, it will be apparent to those skilled in the art that these concepts may be practiced without these specific details. In some instances, well known, structures, techniques and components are shown in block diagram form to avoid obscuring the described concepts and features.

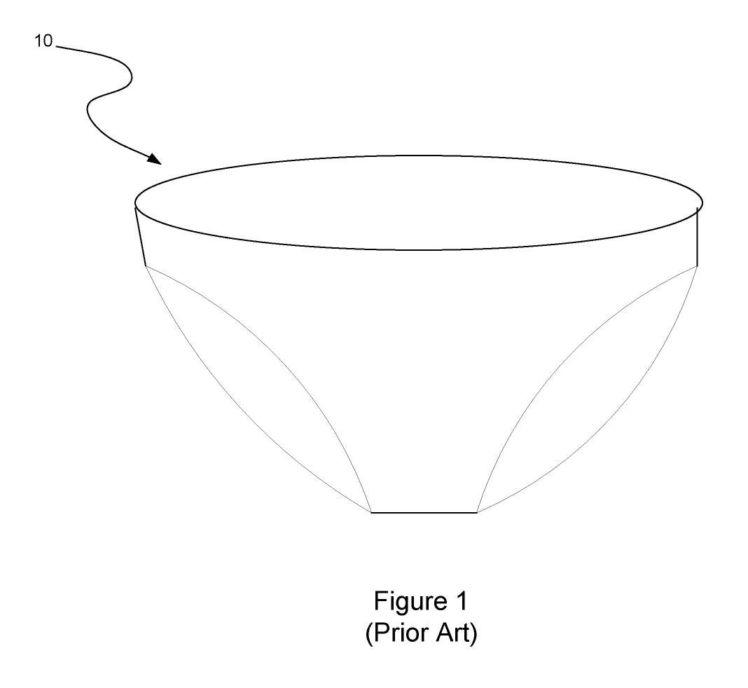

FIG. 1 is a perspective view of an example of underwear 10 of the prior art. The present invention overcomes the disadvantages of the prior art by providing a detachable gusset pad over the gusset of underwear as exemplified in FIG. 1.

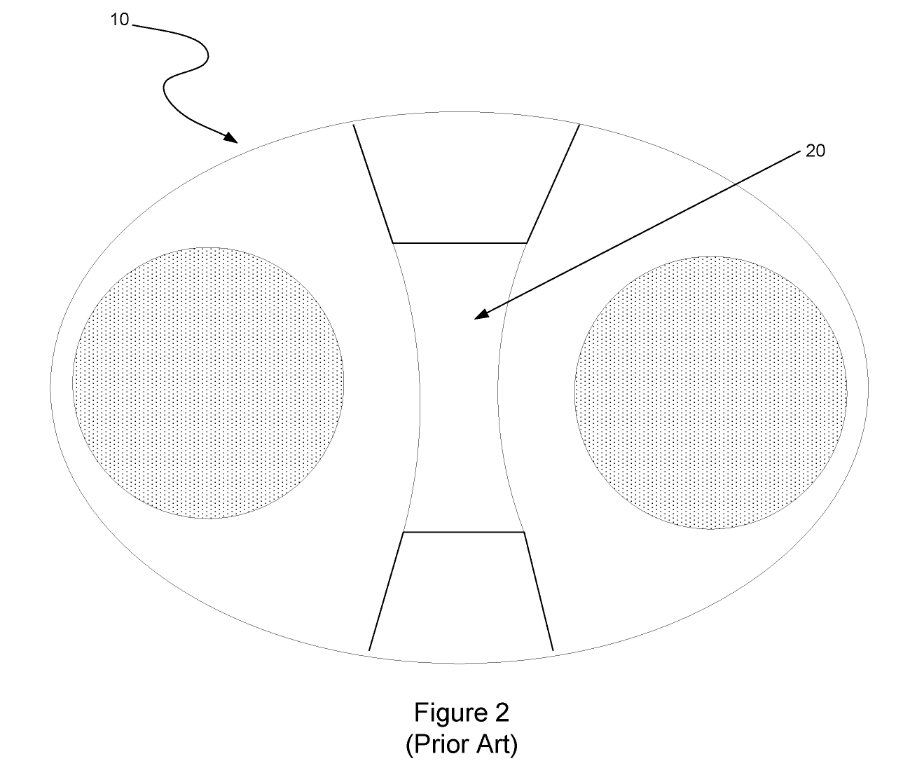

FIG. 2 is a top view of an example of underwear 10 in an embodiment of the prior art including a gusset 20. The gusset 20 is generally located in a central position of the underwear 10.

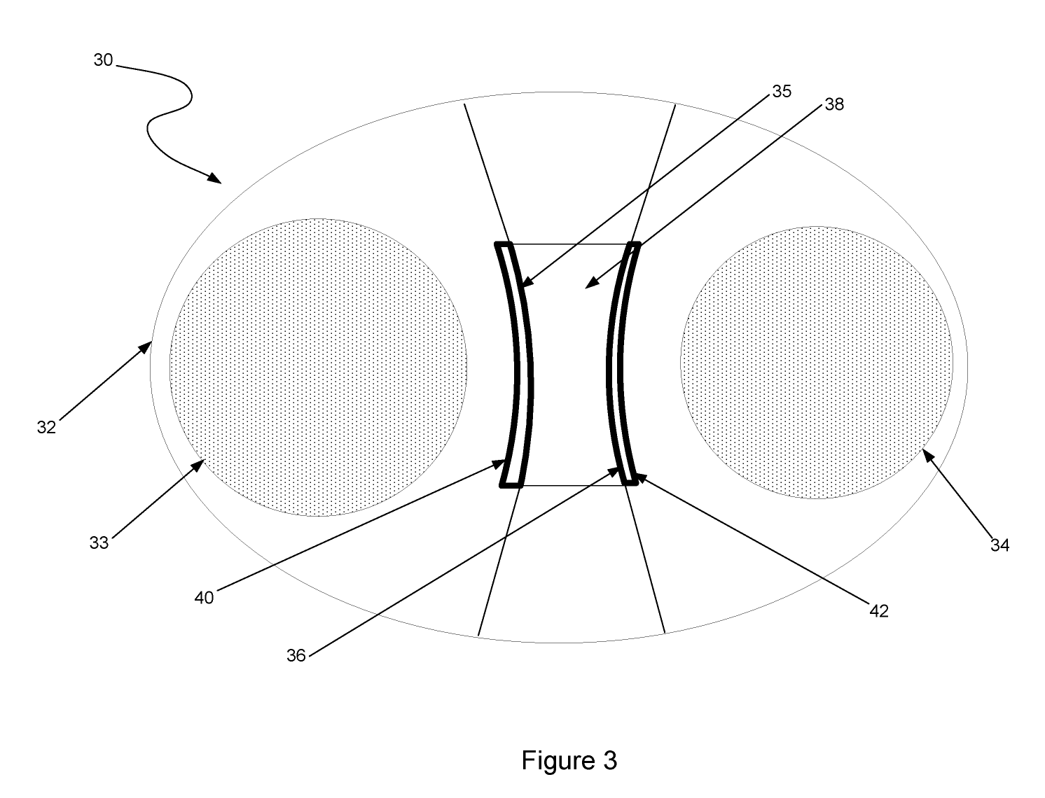

FIG. 3 is a top view of a detachable panel underwear system 30 in an embodiment of the present invention. The underwear system 30 includes three outer edges, a waist band outer edge 32, a first leg outer edge 33 and a second leg outer edge 34. The underwear system 30 further includes a gusset 38 on an inside central area of the underwear system 30. The gusset 38 is disposed centrally between the first leg outer edge 33 and the second leg outer edge 34. The central gusset 38 includes a first seam 35 and a second seam 36. A first Velcro gusset panel 40 is attached to the first seam 35 of the gusset 38 and a second Velcro gusset panel 42 is attached to the second seam 36 of the gusset 38. The Velcro gusset panels 40, 42 may be attached by being sewn onto the first seam 35 and the second seam 36. Alternatively, the Velcro gusset panels 40, 42 may be attached to the first and second seams 35, 36 by glue or other attachment methods know in the art.

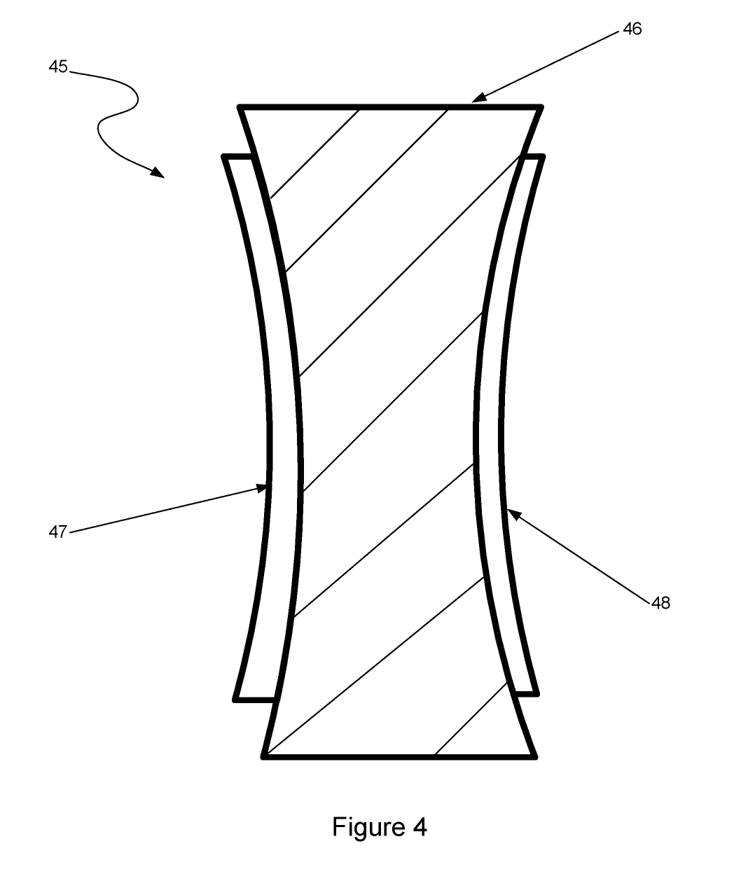

FIG. 4 is a plan view of an exemplary gusset pad 45 in a novel embodiment of the present invention. The gusset pad 45 includes a soft pad 46 and a first Velcro pad panel 47 and a second Velcro pad panel 48. The Velcro pad panels 47, 48 are attached to each side of the gusset pad 45. The Velcro pad panels 47, 48 may be attached to the gusset pad 45 by being sewn onto the soft pad 46, by gluing or other attachment methods know in the art. The soft pad 46 may be made of cotton or other soft absorbent fabric such that discharged material from menstruation and/or incontinence may be absorbed through the soft pad 46.

The gusset pad 45 is disposed opposite the gusset 38 of the underwear system 30 such that the first Velcro gusset panel 40 mates uniformly with the first Velcro pad panel 47 and the second Velcro gusset panel 42 mates uniformly with the second Velcro pad panel 48.

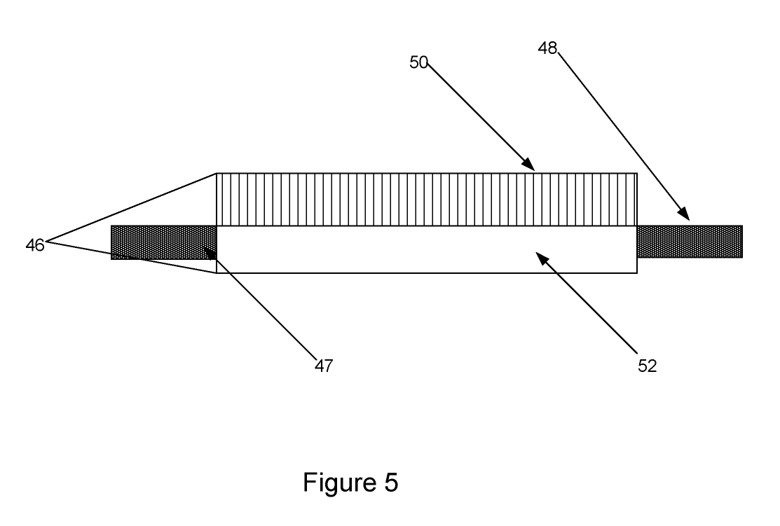

Referring to FIG. 5, the soft pad 46 further includes a first side 50 with the absorbent fabric in contact with the wearer's body and a second side 52 away from the wearer (not shown). The second side 52 may be made of a non-absorbent material such as plastic or non-leaking polyester to prevent the discharge from passing through onto the fabric of the underwear system 30 and onto the wearer's clothes.

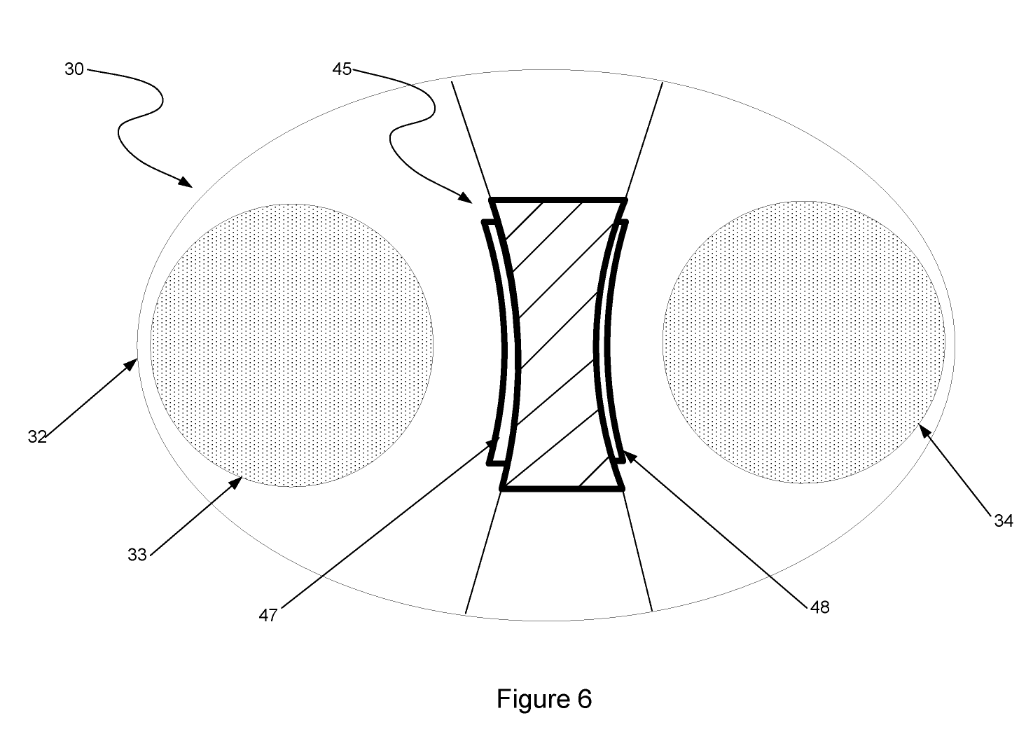

FIG. 6 illustrates the gusset pad 45 attached to the gusset 38 (covered and not shown) with the first Velcro pad panel 47 and the second Velcro pad panel 48 mated over the first and second Velcro gusset panels 40, 42 (not shown). The gusset pad 45 is disposed centrally between the first leg outer edge 33 and the second leg outer edge 34.

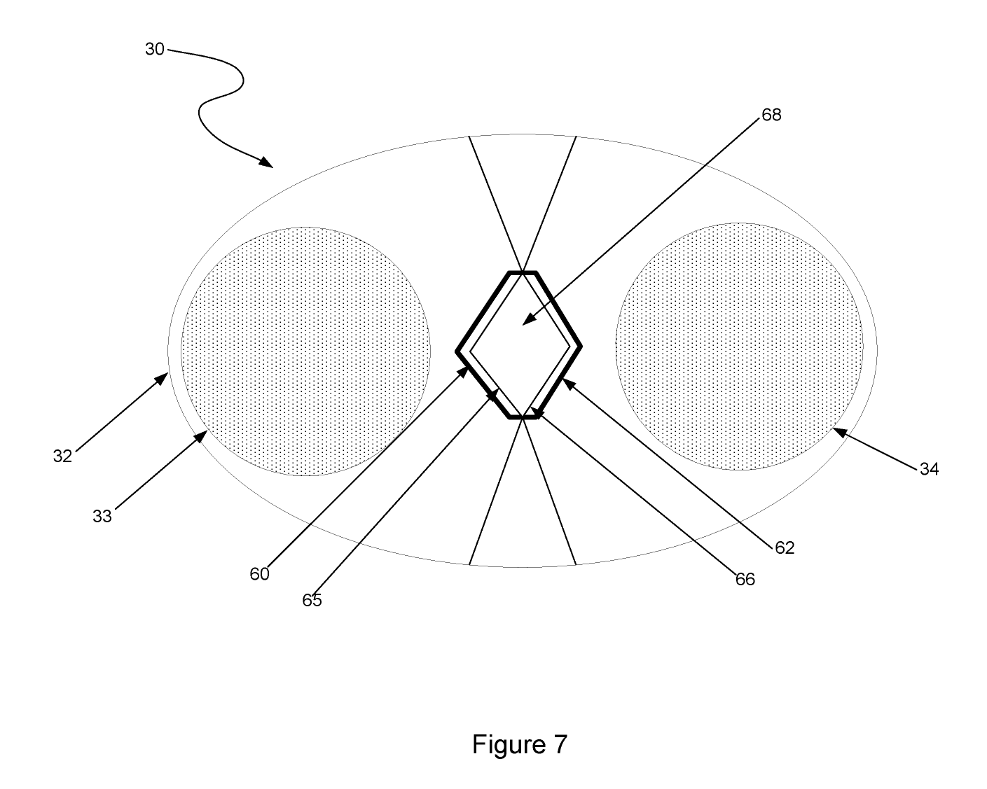

The gusset pad 45 shown in FIGS. 4 and 6 is intended as an example only and not limited to the shape shown in FIGS. 4 and 6. For example, FIG. 7 illustrates a gusset 68 of diamond shape. Other shapes are also possible such as hexagonal shaped gussets. FIG. 7 includes the underwear system 30 and three outer edges, a waist band outer edge 32, a first leg outer edge 33 and a second leg outer edge 34 along with the diamond gusset 68 on an inside central area of the underwear system 30. The gusset 68 is disposed centrally between the first leg outer edge 33 and the second leg outer edge 34. The central gusset 68 includes a first seam 65 and a second seam 66. A first Velcro gusset panel 60 is attached on the first seam 65 of the gusset 68 and a second Velcro gusset panel 62 is attached to the second seam 66 of the gusset 68. The Velcro gusset panels 60, 62 may be attached by being sewn, glued or other attachment methods know in the art onto the first seam 65 and the second seam 66.

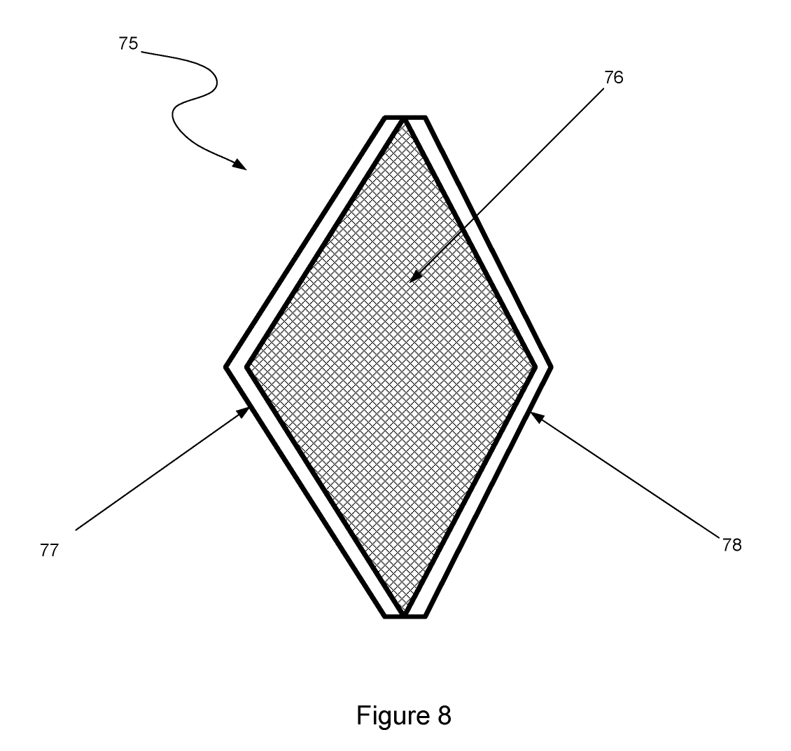

FIG. 8 is a plan view of another exemplary gusset pad 75 in a novel embodiment of the present invention. The gusset pad 75 includes a soft pad 76 and a first Velcro pad panel 77 and a second Velcro pad panel 78. The Velcro pad panels 77, 78 are attached to each side of the gusset pad 75. The Velcro pad panels 77, 78 may be attached to the gusset pad 75 by being sewn onto the soft pad 76, by gluing or other attachment methods know in the art. The soft pad 76 as discussed above may be made of cotton or other soft absorbent fabric such that discharged material from menstruation and/or incontinence may be absorbed through the soft pad 76. The soft pad 76 further includes a top side with the absorbent fabric in contact with the wearer's body and a second side away from the wearer (not shown) in similar fashion as shown in FIG. 5. The second side may be made of a non-absorbent material such as plastic or non-leaking polyester to prevent the discharge from passing through the underwear system 30 and onto the wearer's clothes.

The gusset pad 75 is disposed opposite the gusset 68 of the underwear system 30 such that the first Velcro gusset panel 60 mates uniformly with the first Velcro pad panel 77 and the second Velcro gusset panel 62 mates uniformly with the second Velcro pad panel 78.

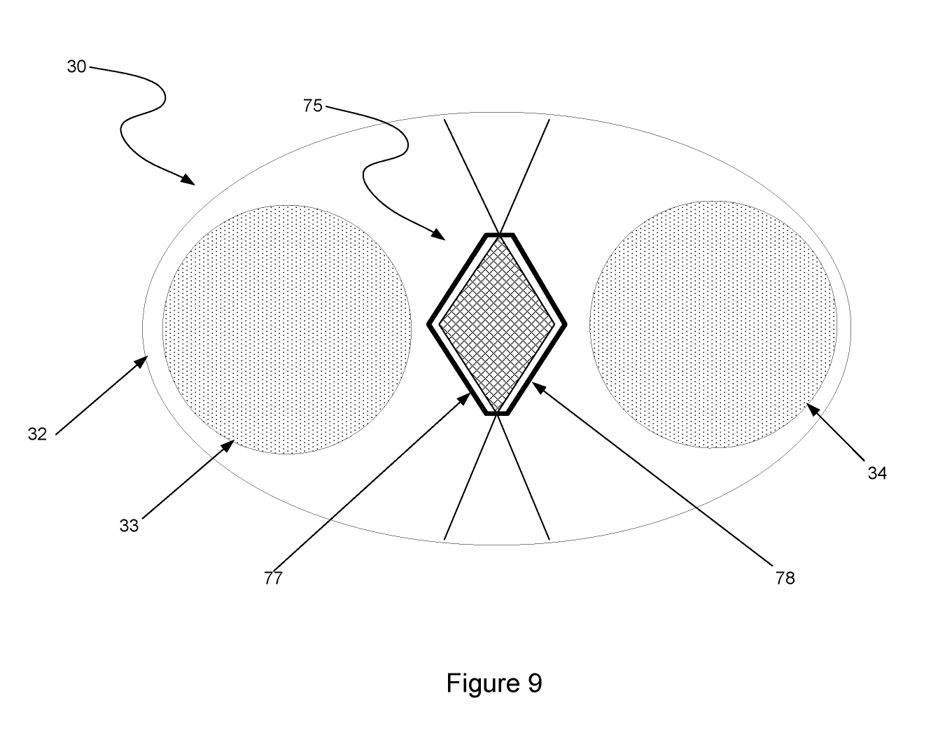

FIG. 9 illustrates the gusset pad 75 attached to the gusset 68 (not shown) with the first Velcro pad panel 77 and the second Velcro pad panel 78 mated over the first and second Velcro gusset panels 60, 62 (not shown). The gusset pad 75 is disposed centrally between the first leg outer edge 33 and the second leg outer edge 34.

The gusset pads 45 and 75 may be of any shape and may be made of various soft fabrics, colors and design in order to conform to a desired fashion. The gusset pads 45 and 75 may be available separate from the underwear system 30 allowing the gusset pads to be replaced for washing or disposal.

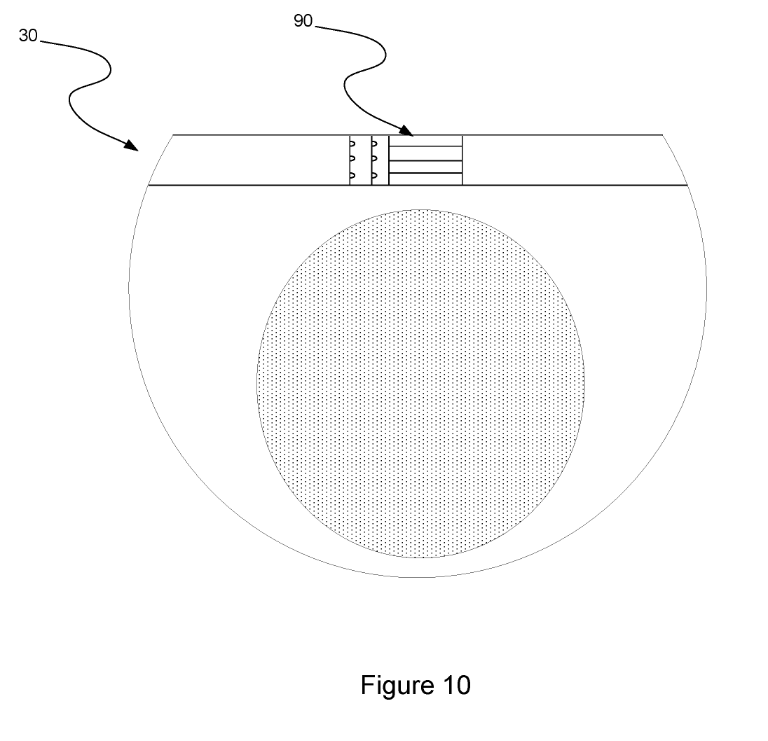

Referring now to FIG. 10 is a perspective side view of the detachable panel underwear system 30 in an embodiment that includes an adjustable clasp 90. The adjustable clasp 90 may be, for example a hook and eye type fastener that includes several fastening positions available for the wearer.

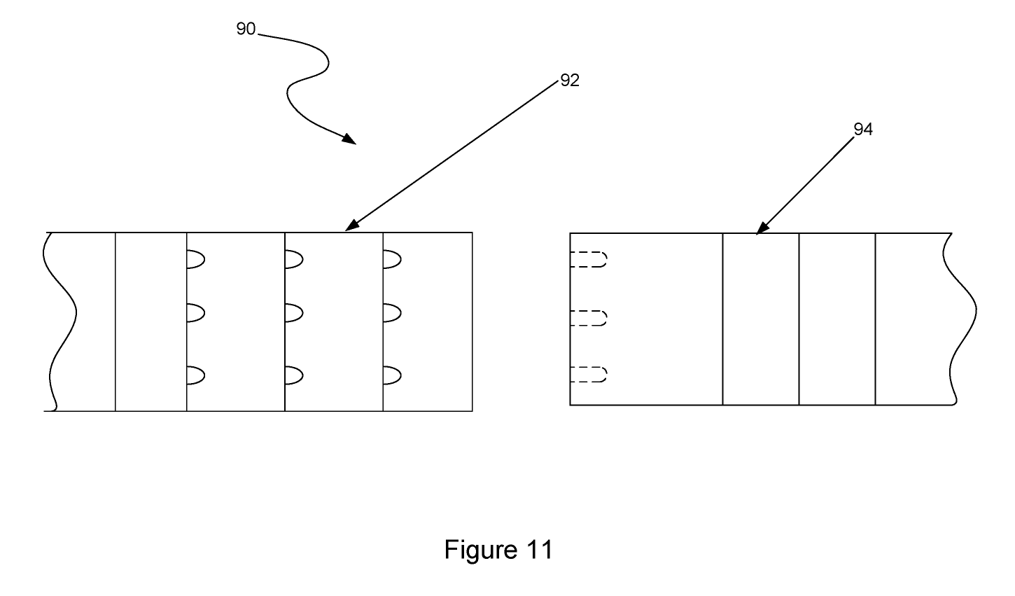

FIG. 11 is an isolation view of the adjustable clasp 90 that includes a hook portion 94 and an eye portion 92 with adjustable positions for the hook 94.

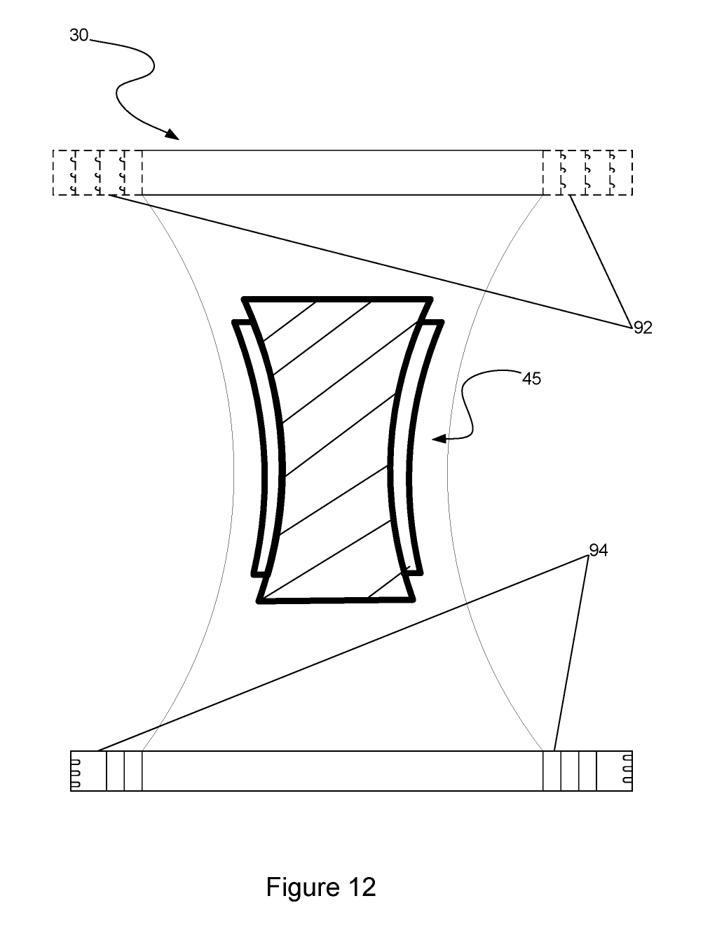

The adjustable clasp 90 may be included on one side of the underwear system 30 as shown in FIG. 10 or alternatively, as shown in FIG. 12, the hook 94 and eye 92 portions of clasp 90 may be included on two sides of the underwear system 30.

Although a hook and eye type fastener is shown in the examples of FIGS. 10-12, any fastening system may be employed including Velcro, single hook fasteners and the like. FIGS. 10-12 are intended as examples only to illustrate the embodiments of the invention.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

I claim:

1. A detachable panel underwear system comprising:
a fabric undergarment including three outer edges, the three outer edges including a waist band outer edge, a first leg outer edge and a second leg outer edge,
a central gusset; and
a detachable gusset pad,
wherein the central gusset is located between the first leg outer edge and the second leg outer edge and the detachable gusset pad is disposed to attach over the central gusset.

2. The detachable panel underwear system of claim 1, wherein the detachable gusset pad includes a first side and a second side wherein the first side includes an absorbent material and the second side includes a non-absorbent material.

3. The detachable panel underwear system of claim 2, wherein the detachable gusset pad is wearable by women during a menstrual period and is replaceable after absorbent use.

4. The detachable panel underwear system of claim 2, wherein the detachable gusset pad is washable.

5. The detachable panel underwear system of claim 2, wherein the detachable gusset pad is disposable.

6. The detachable panel underwear system of claim 1, wherein the central gusset includes a first seam and a second seam wherein the first seam includes a first Velcro gusset panel and the second seam includes second Velcro gusset panel.

7. The detachable panel underwear system of claim 6, wherein the detachable gusset pad includes a soft pad, a first Velcro pad panel and a second Velcro pad panel.

8. The detachable panel underwear system of claim 7, wherein the first Velcro pad panel attaches uniformly to the first Velcro gusset panel and the second Velcro pad panel attaches uniformly to the second Velcro gusset panel.

9. The detachable panel underwear system of claim 1, wherein the fabric undergarment includes an adjustable clasp adjacent to the waist band outer edge.

10. An underwear system comprising:
a fabric undergarment including three outer edges, the three outer edges including a waist band outer edge, a first leg outer edge and a second leg outer edge,
a central gusset;
a detachable gusset pad; and
a clasp adjacent to the waist band outer edge,
wherein the central gusset is located between the first leg outer edge and the second leg outer edge and the detachable gusset pad is disposed to attach over the central gusset.

11. The underwear system of claim 10, wherein the detachable gusset pad includes a first side and a second side wherein the first side includes an absorbent material and the second side includes a non-absorbent material.

12. The underwear system of claim 11, wherein the detachable gusset pad is wearable by women during a menstrual period and is replaceable after absorbent use.

13. The underwear system of claim 11, wherein the detachable gusset pad is washable.

14. The underwear system of claim 11, wherein the detachable gusset pad is disposable.

15. The underwear system of claim 10, wherein the central gusset includes a first seam and a second seam wherein the first seam includes a first Velcro gusset panel and the second seam includes second Velcro gusset panel.

16. The underwear system of claim 15, wherein the detachable gusset pad includes a soft pad, a first Velcro pad panel and a second Velcro pad panel, and wherein the first Velcro pad panel attaches uniformly to the first Velcro gusset panel and the second Velcro pad panel attaches uniformly to the second Velcro gusset panel.

17. The underwear system of claim 10, wherein the clasp is adjustable.

18. The underwear system of claim 17 wherein the clasp is a hook and eye fastener including a plurality of positions to insert the hook.

19. The underwear system of claim 18, wherein the clasp is on a side of the fabric undergarment.

20. The underwear system of claim 18, wherein the clasp includes a first clasp and a second clasp, the first and second clasps are adjacent to the waist band outer edge and the first clasp is on an opposite side of the second clasp, and the first clasp and the second clasp are adjustable.
